(19) United States
(12) Patent Application Publication
(10) Pub. No.: US 2003/0216364 A1
(43) Pub. Date: Nov. 20, 2003

(54) Dermatological Formulation

(76) Inventor: Keith Arthur Johnson, Durham, NC (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/344,797

(22) PCT Filed: Aug. 13, 2001
(86) PCT No.: PCT/US01/25334

(51) Int. Cl. A61K 31/573

(52) U.S. Cl. 514/179

(57) ABSTRACT

A topical formulation including a solvent, an occlusive agent, a surfactant system, an androstane steroid compound of formula (I) wherein R¹ represents a fluoro-, chloro- or bromo-methyl group or a 2′-fluoroethyl group, R² represents a group COR⁶ where R⁶ is a C₁₋₃ alkyl group or OR² and R³ together form a 16α, 17α-isopropylidenedioxy group; R³ represents a hydrogen atom, a methyl group (which may be in either the α- or β-configuration) or a methylene group; R⁴ represents a hydrogen, chlorine or fluorine atom; R⁵ represents a hydrogen or fluorine atom and the symbol --- represents a single or double bond, and the balance being water.

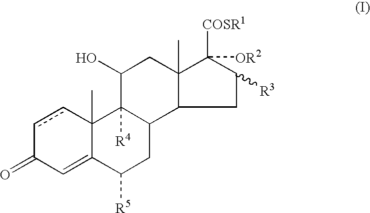

### FIELD OF THE INVENTION

The present invention is generally directed to a dermatological formulation for topical application comprising an androstane steroid compound, where the formulation has improved stability and often also improved vasoconstrictor potency.

Table of Abbreviations

| | |
|---|---|
| BP | British Pharmacopoeia |
| C | centigrade |
| FP | fluticasone propionate |
| HLB | hydrophile-lipophile balance |
| NF | National Formulary |
| % | percent |
| PEG | polyethylene glycol |
| PG | propylene glycol |
| USP | United States Pharmacopoeia |
| VC | vasoconstrictor |
| w/w | weight/weight |

### BACKGROUND OF THE INVENTION

Androstane steroid compounds are a type of anti-inflammatory steroid and are described in U.S. Pat. No. 4,335,121 to Phillipps et al. (Assignors to Glaxo Group Limited). Fluticasone propionate, an androstae steroid compound that is in accordance with the Phillips, et al. patent, has very desirable anti-inflammatory, anti-pruitic, and vasoconstrictive properties.

A fluticasone propionate lotion is described in International Publication No. WO 00/24401, published May 4, 2000, to Dow et al. (Assignors to Glaxo Group Limited). This International Publication states that the fluticasone propionate lotion shows increased vasoconstrictor potency of fluticasone propionate over fluticasone propionate cream formulations but at a decreased concentration of occlusive agent under about 10.0 w/w %. It is known that addition of an occlusive agent, such as mineral oil or paraffin, increases the vasoconstrictor potency of the topical steroid, but that can reduce the aesthetic appeal due to imparting an undesirable oily or greasy feel to the skin. Furthermore, high concentrations of occlusive agents can cause the formulation, which is an oil-in-water emulsion, to be unstable and invert to a water-in-oil emulsion that has the greasy feel.

The disclosures of all mentioned patents and published patent applications are incorporated here by reference.

While the above-noted prior patent, International Publication, and commercial CUTIVATE® cream have attempted to solve the problem of having high vasoconstrictor potency, while not having the instability problem of the emulsion inverting from oil-in-water to being water-in-oil which causes a greasy feel to the skin, it has still been desirable to find a way so that the topical product can have a high % of occlusive agent in order to increase the vasoconstrictor potency but still be stable and not invert to being a water-in-oil emulsion. Applicant's invention is a significant advancement in such a topical product containing fluticasone propionate and/or another androstane steroid compound as the active medicament agent.

### SUMMARY AND OBJECTS OF THE INVENTION

Accordingly, the present invention provides a stable oil-in-water emulsion topical formulation comprising a solvent, an occlusive agent, a surfactant system, water, and an androstane steroid compound of the formula

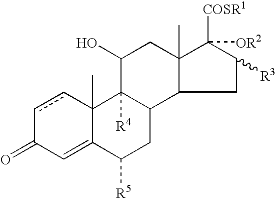

wherein R¹ represents a fluoro-, chloro- or bromo-methyl group or a 2′-fluoroethyl group, R² represents a group COR⁶ where R⁶ is a C₁₋₃ alkyl group or OR² and R³ together form a 16α,17α-isopropylidenedioxy group; R³ represents a hydrogen atom, a methyl group (which may be in either the α- or β-configuration) or a methylene group; R⁴ represents a hydrogen, chlorine or fluorine atom; R⁵ represents a hydrogen or fluorine atom and the symbol --- represents a single or double bond. The androstane steroid compound is present in a w/w % amount from about 0.005% to about 0.05%. The solvent is present in a w/w % amount from about 5% to about 30.0%. The occlusive agent is present in a w/w % amount of at least about 10.1% up to about 50.0%. The surfactant system is selected from at least one surfactant, wherein the surfactant system has a HLB value ranging from about 7.0 to about 10.9, and the surfactant system is present in a w/w % amount from about 0.25 to about 10.0. Suitably, the androstane steroid compound comprises fluticasone, or a pharmaceutically acceptable salt or ester thereof.

Additionally, the present invention provides a process for preparing a topical formulation, that is a stable oil-in-water emulsion, comprising mixing a solvent, an occlusive agent, a surfactant system, water, and an androstane steroid compound of the formula

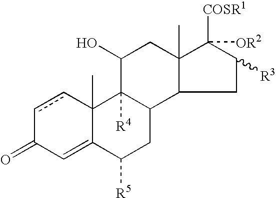

wherein R¹ represents a fluoro-, chloro- or bromo-methyl group or a 2′-fluoroethyl group, R² represents a group COR⁶ where R⁶ is a C₁₋₃ alkyl group or OR² and R³ together form a 16α,17α-isopropylidenedioxy group; R³ represents a hydrogen atom, a methyl group (which may be in either the α- or β-configuration) or a methylene group; R⁴ represents a hydrogen, chlorine or fluorine atom; R⁵ represents a hydrogen or fluorine atom and the symbol --- represents a single or double bond. The androstane steroid compound is present in a w/w % amount from about 0.005% to about 0.05%. The solvent is present in a w/w % amount from about 5% to about 30.0%. The occlusive agent is present in a w/w % amount of at least about 10.1% up to about 50.0%. The surfactant system is selected from at least one surfactant, wherein the surfactant system has a HLB value ranging from about 7.0 to about 10.9, and the surfactant system is present in a w/w % amount from about 0.25 to about 10.0%. The mixing of the aqueous and non-aqueous phases is performed with heat followed by cooling to obtain a stable oil-in-water emulsion. Suitably, the androstane steroid compound comprises fluticasone, or a pharmaceutically acceptable salt or ester thereof.

Furthermore, the present invention provides a process for topically treating a skin condition, such as corticosteroid-responsive dermatosis, atropic dermatitis, inflammation, eczema, erythema, papulation, scaling, erosion, oozing, crusting, pruritis, impetigo, epidermalysis bullosa, psoriasis, erythema, hidradenitis suppurative, warts, diaper rash, jock itch, and combinations thereof, by topically applying a stable oil-in-water emulsion topical formulation comprising a solvent, an occlusive agent, a surfactant system, water, and an androstane steroid compound of the formula

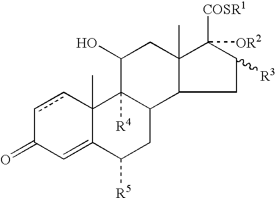

wherein R¹ represents a fluoro-, chloro- or bromo-methyl group or a 2′-fluoroethyl group, R² represents a group COR⁶ where R⁶ is a C₁₋₃ alkyl group or OR² and R³ together form a 16α,17α-isopropylidenedioxy group; R³ represents a hydrogen atom, a methyl group (which may be in either the α- or β-configuration) or a methylene group; R⁴ represents a hydrogen, chlorine or fluorine atom; R⁵ represents a hydrogen or fluorine atom and the symbol --- represents a single or double bond. The androstane steroid compound is present in a w/w % amount from about 0.005% to about 0.05 w/w %. The solvent is present in a w/w % amount from about 5% to about 30.0%. The occlusive agent is present in a w/w % amount of at least about 10.1% up to about 50.0%. The surfactant system is selected from at least one surfactant, wherein the surfactant system has a HLB value ranging from about 7.0 to about 10.9, and the surfactant system is present in a w/w amount from about 0.25 to about 10.0%. Suitably, the androstane steroid compound comprises fluticasone, or a pharmaceutically acceptable salt or ester thereof.

Therefore, it is an object of the present invention to provide a topical formulation having a high % of occlusive agent, and thus, a high vasoconstrictor potency, yet the formulation also has stability and does not invert to being a water-in-oil emulsion.

Consequently, it is an advantage of the present invention that the topical formulation, even though it has a high % of occlusive agent, does not impart a greasy feel to the skin.

Some of the objects and advantages of the present invention having been stated above, other objects will become evident as the description proceeds, when taken in connection with the accompanying Laboratory Examples as best described below.

### DETAILED DESCRIPTION OF THE INVENTION

The topical formulation of the present invention is a stable oil-in-water emulsion. Due to careful choosing of the surfactant system to have a particular HLB value in a range from about 7.0 to about 10.9, the formulation can have an increased amount of occlusive agent. Such increase results in improved VC potency, often with a VC ranking of I or II. The formulation is free of the prior art problem of an increased amount of occlusive agent causing a formulation to be unstable and, thus, invert to a water-in-oil emulsion that imparts an undesirable greasy feel to the skin. A water-in-oil emulsion is also disadvantageous in that it has poor content uniformity and is lacking in pharmaceutical elegance (i.e., fails to meet appearance requirements).

Suitable compounds useful as the active medicament agent in the topical formulation of the present invention are the androstane steroid compounds of formula (I)

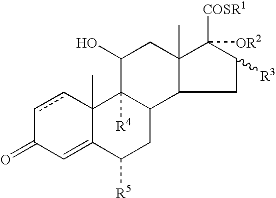

wherein R¹ represents a fluoro-, chloro- or bromo-methyl group or a 2′-fluoroethyl group, R² represents a group COR⁶ where R⁶ is a C₁₋₃ alkyl group or OR² and R³ together form a 16α,17α-isopropylidenedioxy group; R³ represents a hydrogen atom, a methyl group (which may be in either the α- or β-configuration) or a methylene group; R⁴ represents a hydrogen, chlorine or fluorine atom; R⁵ represents a hydrogen or fluorine atom and the symbol --- represents a single or double bond.

Compounds of formula (I) which have good anti-inflammatory activity coupled with minimal hypothalamuspituitary-adrenal-suppressive activity when applied topically include, but are not limited to, 1,4-dienes in which R¹ is chloro- or fluoro-methyl, R⁴ and R⁵ are fluorine and in particular those in which R³ is α-methyl.

Especially suitable compounds of formula (I) in view of their good topical anti-inflammatory activity and favorable ratio of topical anti-inflammatory activity to undesired systemic activity include, but are not limited to: S-chloromethyl 9α-fluoro-11β-hydroxy-16α-methyl-3-oxo-17α-propionyloxyandosta-1,4-diene-17β-carbothioate; S-chloromethyl 9α-fluoro-11β-hydroxy-16-methylene-3-oxo-17α-propionyloxyandosta-1,4-diene-17β-carbothioate; S-fluoromethyl 6α,9α-difluoro-11β-hydroxy-16α,17α-isopropylidenedioxy-3-oxoandrosta-1,4diene-17β-carbothioate; S-fluoromethyl 6α,9α-difluoro-11β-hydroxy-16α-methyl-3-oxo-17α-propionyloxyandosta-1,4-diene-17β-carbothioate; S-chloromethyl 6α,9α-difluoro-11β-hydroxy-16α-methyl-3-oxo-17α-propionyloxyandosta-1,4-diene-17β-carbothioate, and combinations thereof. The last compound is especially preferred in view of its particularly favorable ratio and in addition minimal skin atrophy.

In particular, a very suitable androstane steroid compound is fluticasone, or a pharmaceutically acceptable salt or ester thereof, especially fluticasone propionate. When the androstane steroid compound is FP, then R¹ represents fluoromethyl, R² represents COR⁶ where R⁶ represents C₂H₅, and each of R⁴ and R⁵ represents fluoro. The chemical formula of FP is [(6α,11β,16α,17α)-6,9,-difluoro-11-hydroxy-16-methyl-3-oxo-17-(1-oxopropoxy) androsta-1,4-diene-17-carbothiotic acid, S-fluoromethyl ester].

The androstane steroid compound should be present in the topical formulation in an amount ranging from about 0.005 to about 0.05 w/w %, particularly from about 0.005 to about 0.10 w/w %, and more particularly from about 0.01 to about 0.05 w/w %.

The one or more various solvents that may be present in the topical formulation comprise various short chain alcohols including, but not limited to, ethyl alcohol, propylene glycol, triacetin, hexylene glycol, and combinations thereof. The solvent may be present in an amount ranging from about 5.0 to about 30.0 w/w %, particularly from about 7.0 to about 20.0 w/w %, and more particularly from about 10.0 to about 15.0 w/w %. More particularly, the solvent is PG in an amount ranging from about 5.0 to about 15.0 w/w %.

Suitable occlusive agents that may be present in the topical formulation include, but are not limited to, petrolatum, microcrystalline wax, dimethicone, beeswax, mineral oil, squalane, liquid paraffin, shea butter, carnauba wax, SEPIGEL® (a blend of isoparaffin/polyacrylamide/laureth-7), and combinations thereof. The occlusive agent should be present in an amount of at least about 10.1 w/w %. In particular, the amount of occlusive agent may range from about 15.00 to about 50.0 w/w %, more particularly from about 20.0 to about 45.0 w/w %, and even more particularly from about 25.0 to about 42.5 w/w %. More particularly, at least two of the occlusive agents are present, with one of them being microcrystalline wax in an amount of at least 10.0 w/w %, more particularly at least 10.1 w/w %.

The surfactant system comprises at least one surfactant and exhibits a HLB value in a range from about 7.0 to about 10.9, particularly from about 7.5 to about 10.5, and more particularly from about 8.0 to about 10.0. The surfactant system may be present in the formulation in an amount ranging from about 0.25 to about 10.0 w/w %, particularly from about 0.40 to about 9.0 w/w %, and more particularly from about 2.0 to about 4.0 w/w %, with 3 w/w % being optimal. Suitable surfactants include, but are not limited to, CETOMACROGOL® 1000, (Crodor, Inc.) glycerol monostearate, glycerol distearate, glyceryl stearate, polyoxyethylene stearate, a blend of glyceryl stearate and PEG-100 stearate (as ARLACEL 165), polysorbate 40, polysorbate 60, polysorbate 80, CETETH-20®, sorbitan monopalimate, sorbitan monostearate, sorbitan monooleate, and combinations thereof. Especially suitable surfactant systems are as follows.

| HBL | Surfactants System (Total is 3 w/w % of Formulation) |
|---|---|
| 8 | ARLACEL 165* (1.74 w/w %)/Cerasynt³⁰ SD (1.26 w/w %) |
| 9 | Polysorbate 60 (1.26 w/w %)/sorbitan monostearate |
| | (1.74 w/w %) |
| 9 | ARLACEL 165* (2 w/w %)/sorbitan monostearate (1 w/w %) |
| 10 | Polysorbate 80 (1.6 w/w %)/sorbitan monooleate (1.4 w/w %) |
| 10 | Polysorbate 60 (1.56 w/w %)/sorbitan monostearate |
| | (1.44 w/w %) |
| 10 | Polysorbate 40 (1.11 w/w %)/sorbitan monopalmitate |
| | (1.89 w/w %) |

*Glyceryl stearate and PEG 100 stearate
*Glyceryl stearate (self dispersing)

The topical formulation comprises an androstane steroid compound, a solvent, an occlusive agent, and a surfactant system, in the amounts noted, with the balance being water.

Various optional ingredients may also be present in the inventive topical formulation. These are carriers (such as water or mineral oil), skin conditioners (such as lanolin, glycerine, cholesterol, cetostearyl alcohol, dimethicone PEG 100, PEG 200, PEG 300, PEG 400 or isopropylmyristate), buffers (such as sodium citrate/citric acid, dibasic sodium phosphate/citric acid, or monobasic sodium phosphate/citric acid), or preservatives (such as imidurea, methylparaben, or propylparaben).

The inventive topical formulation is useful for treatment of various skin conditions. The formulation may be topically applied to the affected area of the skin as a cream, a lotion, an ointment, and the like. Representative skin conditions include, but are not limited to, corticosteroid-responsive dermatosis, atropic dermatitis, inflammation, eczema, erythema, papulation, scaling, erosion, oozing, crusting, pruritis, impetigo, epidermalysis bullosa, psoriasis, erythema, hidradenitis suppurative or warts. The treatment regimen may be varied from patient to patient and condition to condition. In general, the formulation is to be applied once or twice per day to a treatment area.

The formulation of the present invention may be manufactured in a conventional manner by mixing the various ingredients at elevated temperatures, typically from about 40° C. to about 80° C., followed by cooling to achieve a smooth, homogenous oil-in-water emulsion, which is stable and does not invert to a water-in-oil emulsion. Typically, three phases are prepared: an aqueous phase, a non-aqueous phase, and a medicament slurry. The aqueous phase and the non-aqueous phase are heated (about 40° C. to about 80° C.), while the medicament slurry is maintained near room temperature (about 15° C. to about 40° C.). Next, the three phases are combined in a mixing vessel where the temperature can be controlled, and the three phases are mixed so that the non-aqueous phase is emulsified and the medicament particles are uniformly dispersed. The intensity of the mixing is variable. The emulsion is cooled while the mixing continues. When the emulsion reaches the desired temperature (usually about 15° C. to about 30° C.), the emulsion is removed from the mixing vessel and filled into the appropriate container/closure system.

The following Laboratory Examples are intended merely to illustrate the formulation of the present invention and are not to be construed as limiting the scope of the invention. Unless indicated otherwise, all weight percentages are based on the total weight of the formulation. Also, the vasoconstrictor evaluations were measured using a VC assay (McKenzie and Stoughton, Arch. Dermatol., 86, 608 (1962)), using a scale of I, II, III, IV, and V, where I is the highest potency ranking and V is the lowest potency ranking. The VC assay according to McKenzie and Stoughton is a standard dermatological assay used to predict the potency of corticosteroid formulations. Potency is related to both side effect potential and efficacy in the treatment of mild to severe skin conditions, as mentioned above. Reactions of particular concern include skin thinning (atrophy, including telangectasia), and adrenal axis suppression, which can occur oftener under occlusions or when higher potency corticosteroids are employed.

### LABORATORY EXAMPLES

### Example 1

Three topical 0.05 w/w % fluticasone propionate formulations were prepared, where the first formulation and the second formulation were comparisons, and the third formulation was in accordance with the present invention. These formulations had the following respective ingredients, indicated in w/w %, and the following respective characteristics of VC and HLB.

| | Formulation | |
|---|---|---|
| | | Cream 2 |
| Ingredient (and characteristics | Cream 1 | (Preferred |
| of VC and HLB) | (Comparison) | Embodiment) |
| Fluticasone propionate, micronized | 0.05% | 0.05% |
| Propylene glycol, USP | 10.0% | 10.0% |
| Microcrystalline wax, NF | 10.0% | 10.0% |
| Cetostearyl alcohol, NF | 2.0% | 2.0% |
| Liquid paraffin, BP | 40.0% | 32.5% |
| Isopropyl myristate, NF | 5.0% | 7.5% |
| ARLACEL 165* | 3.0% | 2.0% |
| Sorbitan monostearate, NF | — | 1.0% |
| Dimethicone 360, NF | — | 2.5% |
| Imidurea, NF | 0.2% | 0.2%5 |
| Dibasic sodium phosphate, USP | 0.06% | 0.06% |
| Citric acid, Hydrous, USP | 0.05% | 0.05% |
| Purified water, USP | to 100.0% | to 100.0% |
| HLB value | 11 | 9 |

*Glyceryl stearate and PEG 100 stearate

Discussion of cream 1 (comparison). Cream 1 was an old formulation for CUTIVATE® cream. Although the potency was high, the presence of 10.0 w/w % microcrystalline wax plus 40.0 w/w % liquid paraffin, for a high amount of 50.0 w/w % for the occlusive agent, made cream 1 very unstable. Cream 1 inverted from an oil-in-water emulsion to a water-in-oil emulsion, Hence, cream 1 was not suitable for marketing because of the phase inversion and because it lacked homgeneity. Moreover, cream 1 imparted greasy feel to the skin, which is undesirable. The HLB value of the surfactant system (glyceryl stearate and PEG-100 stearate, which blend is sold as ARALCEL 165) in cream 1 was 11.

Discussion of Cream 2. On the other hand, cream 2, which is in accordance with the present invention, has both a high level of occlusive agent and excellent stability as an oil-in-water emulsion over long periods of time and not inverting to a water-in-oil emulsion. It is noted that cream 2 contained a high amount of 42.5 w/w % of occlusive agent, namely 10.0 w/w % of microcrystalline wax plus 32.5 w/w % of liquid paraffin. Also, even though cream 2 had a high amount of occlusive agent, cream 2 still had an excellent stability, and it remained as an oil-in-water emulsion. The HLB value of the surfactant system in cream 2 (glyceryl stearate and PEG-100 stearate, which blend is sold as ARLACEL 165, and also sorbitan monostearate) was chosen to have a particular HLB value. More specifically, the HLB value of the surfactant system in cream 2 was approximately 9.

It will be understood that various details of the invention may be changed without departing from the scope of the invention. Furthermore, the above description is for the purpose of illustration only, and not for the purpose of limitation—the invention being defined by the claims.

What is claimed is:

1. A stable oil-in-water emulsion topical formulation comprising:
(a) about 5 to about 30 w/w % of a solvent;
(b) about 10.1 to about 50.0 w/w % of an occlusive agent;
(c) about 0.25 to about 10.0 w/w % of a surfactant system with an HLB value from about 7.0 to about 10.9;
(d) about 0.005 to about 0.05 w/w % of an androstane steroid compound of the formula 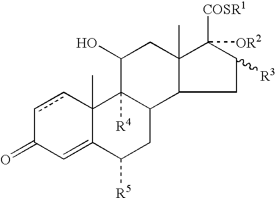
wherein R¹ represents a fluoro-, chloro- or bromo-methyl group or a 2′-fluoroethyl group, R² represents a group COR⁶ where R⁶ is a C₁₋₃ alkyl group or OR² and R³ together form a 16α,17α-isopropylidenedioxy group; R³ represents a hydrogen atom, a methyl group (which may be in either the α- or β-configuration) or a methylene group; R⁴ represents a hydrogen, chlorine or fluorine atom; R⁵ represents a hydrogen or fluorine atom and the symbol --- represents a single or double bond; and
(e) the balance being water.

2. The formulation of claim 1, wherein the solvent is selected from the group consisting of ethyl alcohol, propylene glycol, triacetin, hexylene glycol, and combinations thereof.

3. The formulation of claim 1, wherein the occlusive agent is selected from the group consisting of petrolatum, microcrystalline wax, dimethicone, beeswax, mineral oil, squalane, liquid paraffin, shea butter, carnauba wax, a blend of isoparaffin/polyacrylamide/laureth-7, and combinations thereof.

4. The formulation of claim 1, wherein the surfactant system is selected from the group or surfactants consisting of CETOMACROGOL® 1000, BP (polyoxyethylene (20) cetyl ether, HLB=15.7) glycerol monostearate, glycerol distearate, glyceryl stearate, polyoxyethylene stearate, a blend of glyceryl stearate and PEG-100 stearate, polysorbate 40, polysorbate 60, polysorbate 80, CETETH-20®, sorbitan monopalimate, sorbitan monostearate, sorbitan monooleate, and combinations thereof.

5. The formulation of claim 4, wherein the surfactant system is a blend of at least two surfactants selected from the group consisting of (a) a blend of glyceryl stearate and PEG 100 stearate, (b) a blend of polysorbate 60 and sorbitan monostearate, (c) a blend of glyceryl stearate and PEG 100 stearate and sorbitan monostearate, (d) a blend of polysorbate 80 and sorbitan monooleate, (e) a blend of polysorbate 60 and sorbitan monostearate, and (f) a blend of polysorbate 40 and sorbitan monopalmitate.

6. The formulation of claim 5, where the surfactant system has an HLB value selected from the group consisting of 8, 9, and 10.

7. The formulation of claim 1, wherein the androstane steroid compound is selected from the group consisting of 1,4-dienes in which R¹ is selected from the group consisting of chloro-methyl and fluoro-methyl, and R⁴ and R⁵ are fluorine.

8. The formulation of claim 7, where R³ is α-methyl in the 1-4-dienes.

9. The formulation of claim 1, wherein the androstane steroid compound is selected from the group consisting of S-chloromethyl 9α-fluoro-11∃-hydroxy-16α-methyl-3-oxo-17α-propionyloxyandosta-1,4-diene-17β-carbothioate; S-chloromethyl 9α-fluoro-11β-hydroxy-16-methylene-3-oxo-17α-propionyloxyandosta-1,4-diene-17β-carbothioate; S-fluoromethyl 6α,9α-difluoro-11β-hydroxy-16α,17α-isopropylidenedioxy-3-oxoandrosta-1,4-diene-17β-carbothioate; S-fluoromethyl 6α,9α-difluoro-11β-hydroxy-16α-methyl-3-oxo-17α-propionyloxyandosta-1,4-diene-17β-carbothioate; S-chloromethyl 6α,9α-difluoro-11β-hydroxy-16α-methyl-3-oxo-17α-propionyloxyandosta-1,4-diene-17β-carbothioate, and combinations thereof.

10. The formulation of claim 1, wherein the androstane steroid compound is selected from the group consisting of fluticasone, a pharmaceutically acceptable salt of fluticasone, an ester of fluticasone, and combinations thereof.

11. The formulation of claim 10, wherein the androstane steroid compound is fluticasone propionate.

12. The formulation of claim 1, further including another ingredient selected from the group consisting of a carrier, a skin conditioner, a buffer, a preservative, and combinations thereof.

13. The formulation of claim 1, wherein the formulation has a vasoconstrictor potency ranking selected from the group consisting of I and II.

14. A process for preparing stable oil-in-water emulsion topical formulation, said process comprising the steps of:
(I) mixing with heat:
(a) about 5 to about 30 w/w % of a solvent;
(b) about 10.1 to about 50.0 w/w % of an occlusive agent;
(c) about 0.25 to about 10.0 w/w % of a surfactant system with an HLB value from about 7.0 to about 10.9;
(d) about 0.005 to about 0.05 w/w % of an androstane steroid compound of the formula 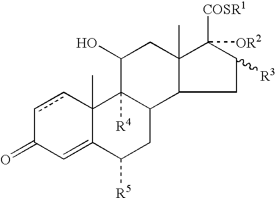
wherein R¹ represents a fluoro-, chloro- or bromo-methyl group or a 2′-fluoroethyl group, R² represents a group COR⁶ where R⁶ is a C₁₋₃ alkyl group or OR² and R³ together form a 16α,17α-isopropylidenedioxy group; R³ represents a hydrogen atom, a methyl group (which may be in either the α- or β-configuration) or a methylene group; R⁴ represents a hydrogen, chlorine or fluorine atom; R⁵ represents a hydrogen or fluorine atom and the symbol --- represents a single or double bond;
(e) the balance being water; and
(II) cooling the resultant of step (I) to obtain a stable oil-in-water emulsion.

15. The process of claim 14, wherein the solvent is selected from the group consisting of ethyl alcohol, propylene glycol, triacetin, hexylene glycol, and combinations thereof.

16. The process of claim 14, wherein the occlusive agent is selected from the group consisting of petrolatum, microcrystalline wax, dimethicone, beeswax, mineral oil, squalane, liquid paraffin, shea butter, carnauba wax, a blend of isoparaffin/polyacrylamide/laureth-7, and combinations thereof.

17. The process of claim 14, wherein the surfactant system is selected from the group or surfactants consisting of CETOMACROGOL® 1000, glycerol monostearate, glycerol distearate, glyceryl stearate, polyoxyethylene stearate, a blend of glyceryl stearate and PEG-100 stearate, polysorbate 40, polysorbate 60, polysorbate 80, CETETH-20®, sorbitan monopalimate, sorbitan monostearate, sorbitan monooleate, and combinations thereof.

18. The process of claim 17, wherein the surfactant system is a blend of at least two surfactants selected from the group consisting of (a) a blend of glyceryl stearate and PEG 100 stearate, (b) a blend of polysorbate 60 and sorbitan monostearate, (c) a blend of glyceryl stearate and PEG 100 stearate and sorbitan monostearate, (d) a blend of polysorbate 80 and sorbitan monooleate, (e) a blend of polysorbate 60 and sorbitan monostearate, and (f) a blend of polysorbate 40 and sorbitan monopalmitate.

19. The process of claim 18, where the surfactant system has an HLB value selected from the group consisting of 8, 9, and 10.

20. The process of claim 14, wherein the androstane steroid compound is selected from the group consisting of 1,4-dienes in which R¹ is selected from the group consisting of chloro-methyl and fluoro-methyl, and R⁴ and R⁵ are fluorine.

21. The process of claim 20, where R³ is α-methyl in the 1-4-dienes.

22. The process of claim 14, wherein the androstane steroid compound is selected from the group consisting of S-chloromethyl 9α-fluoro-11β-hydroxy-16α-methyl-3-oxo-17α-propionyloxyandosta-1,4-diene-17β-carbothioate; S-chloromethyl 9α-fluoro-11β-hydroxy-16-methylene-3-oxo-17α-propionyloxyandosta-1,4-diene-17β-carbothioate; S-fluoromethyl 6α,9α-difluoro-11β-hydroxy-16α,17α-isopropylidenedioxy-3-oxoandrosta-1,4-diene-17β-carbothioate; S-fluoromethyl 6α,9α-difluoro-11β-hydroxy-16α-methyl-3-oxo-17α-propionyloxyandosta-1,4-diene-17β-carbothioate; S-chloromethyl 6α,9α-difluoro-11β-hydroxy-16α-methyl-3-oxo-17α-propionyloxyandosta-1,4-diene-17β-carbothioate, and combinations thereof.

23. The process of claim 14, wherein the androstane steroid compound is selected from the group consisting of fluticasone, a pharmaceutically acceptable salt of fluticasone, an ester of fluticasone, and combinations thereof.

24. The process of claim 23, wherein the androstane steroid compound is fluticasone propionate.

25. The process of claim 14, further including another ingredient selected from the group consisting of a carrier, a skin conditioner, a buffer, a preservative, and combinations thereof.

26. The process of claim 14, wherein the formulation has a vasoconstrictor potency ranking selected from the group consisting of I and II.

27. A process for topically treating a skin condition, said process comprising topically applying a stable oil-in-water emulsion topical formulation comprising:
(a) about 5 to about 30 w/w % of a solvent;
(b) about 10.1 to about 50.0 w/w % of an occlusive agent;
(c) about 0.25 to about 10.0 w/w % of a surfactant system with an HLB value from about 7.0 to about 10.9;
(d) about 0.005 to about 0.05 w/w % of an androstane steroid compound of the formula 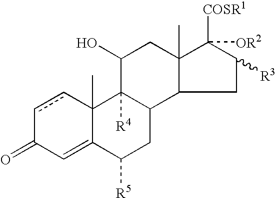
wherein R¹ represents a fluoro-, chloro- or bromo-methyl group or a 2′-fluoroethyl group, R² represents a group COR⁶ where R⁶ is a C₁₋₃ alkyl group or OR² and R³ together form a 16α,17α-isopropylidenedioxy group; R³ represents a hydrogen atom, a methyl group (which may be in either the α- or β-configuration) or a methylene group; R⁴ represents a hydrogen, chlorine or fluorine atom; R⁵ represents a hydrogen or fluorine atom and the symbol --- represents a single or double bond; and
(e) the balance being water.

28. The process of claim 27, wherein the solvent is selected from the group consisting of ethyl alcohol, propylene glycol, triacetin, hexylene glycol, and combinations thereof.

29. The process of claim 27, wherein the occlusive agent is selected from the group consisting of petrolatum, microcrystalline wax, dimethicone, beeswax, mineral oil, squalane, liquid paraffin, shea butter, carnauba wax, a blend of isoparaffin/polyacrylamide/laureth-7, and combinations thereof.

30. The process of claim 27, wherein the surfactant system is selected from the group of surfactants consisting of CETOMACROGOL® 1000, glycerol monostearate, glycerol distearate, glyceryl stearate, polyoxyethylene stearate, a blend of glyceryl stearate and PEG-100 stearate, polysorbate 40, polysorbate 60, polysorbate 80, CETETH-20®, sorbitan monopalimate, sorbitan monostearate, sorbitan monooleate, and combinations thereof.

31. The process of claim 30, wherein the surfactant system is a blend of at least two surfactants selected from the group consisting of (a) a blend of glyceryl stearate and PEG 100 stearate, (b) a blend of polysorbate 60 and sorbitan monostearate, (c) a blend of glyceryl stearate and PEG 100 stearate and sorbitan monostearate, (d) a blend of polysorbate 80 and sorbitan monooleate, (e) a blend of polysorbate 60 and sorbitan monostearate, and (f) a blend of polysorbate 40 and sorbitan monopalmitate.

32. The process of claim 31, where the surfactant system has an HLB value selected from the group consisting of 8, 9, and 10.

33. The process of claim 27, wherein the androstane steroid compound is selected from the group consisting of 1,4-dienes in which R¹ is selected from the group consisting of chloro-methyl and fluoro-methyl, and R⁴ and R⁵ are fluorine.

34. The process of claim 33, where R³ is α-methyl in the 1-4-dienes.

35. The process of claim 27, wherein the androstane steroid compound is selected from the group consisting of S-chloromethyl 9 α-fluoro-11β-hydroxy-16α-methyl-3-oxo-17α-propionyloxyandosta-1,4-diene-17β-carbothioate; S-chloromethyl 9 α-fluoro-11β-hydroxy-16-methylene-3-oxo-17α-propionyloxyandosta-1,4-diene-17β-carbothioate; S-fluoromethyl 6α,9α-difluoro-11β-hydroxy-16α,17α-isopropylidenedioxy-3-oxoandrosta-1,4-diene-17β-carbothioate; S-fluoromethyl 6α,9α-difluoro-11β-hydroxy-16α-methyl-3-oxo-17α-propionyloxyandosta-1,4-diene-17β-carbothioate; S-chloromethyl 6α,9α-difluoro-11β-hydroxy-16α-methyl-3-oxo-17α-propionyloxyandosta-1,4-diene-17β-carbothioate, and combinations thereof.

36. The process of claim 27, wherein the androstane steroid compound is selected from the group consisting of fluticasone, a pharmaceutically acceptable salt of fluticasone, an ester of fluticasone, and combinations thereof.

37. The process of claim 36, wherein the androstane steroid compound is fluticasone propionate.

38. The process of claim 27, further including another ingredient selected from the group consisting of a carrier, a skin conditioner, a buffer, a preservative, and combinations thereof.

39. The process of claim 27, wherein the formulation has a vasoconstrictor potency ranking selected from the group consisting of I and II.

40. The process of claim 27, wherein the skin condition is selected from the group consisting of corticosteroid-responsive dermatosis, atropic dermatitis, inflammation, eczema, erythema, papulation, scaling, erosion, oozing, crusting, pruritis, impetigo, epidermalysis bullosa, psoriasis, erythema, hidradenitis suppurative, warts, diaper rash, jock itch, and combinations thereof.
